(19) United States
(12) Patent Application Publication
(10) Pub. No.: US 2025/0312542 A1
(43) Pub. Date: Oct. 9, 2025

(54) ATOMIZER AND METHOD OF ATOMIZING A LIQUID

(71) Applicants: William Tan, San Gabriel, CA (US); Liat Keng Kang, Singapore (SG)

(72) Inventors: William Tan, San Gabriel, CA (US); Liat Keng Kang, Singapore (SG)

(21) Appl. No.: 18/865,003

(22) PCT Filed: May 12, 2023
(86) PCT No.: PCT/US2023/022150

§ 371 Date: Nov. 12, 2024

Related U.S. Application Data
(60) Provisional application No. 63/341,413, filed on May 12, 2022.

(51) Int. Cl. A61M 11/00; A61M 11/04

(52) U.S. Cl. CPC A61M 11/005; A61M 11/041; A61M 2205/0294; A61M 2205/3368

(57) ABSTRACT

An atomizer including a housing defining a reservoir section, the reservoir section being sized to hold a volume of a liquid in reserve; at least one piezoelectric element coupled to the housing; a mesh coupled to the at least one piezoelectric element; and a heater disposed in the housing and outside of the reservoir section, the heater including a heating face oriented substantially opposing to and spaced apart from the mesh, wherein the mesh and the heating face define an interfacial region therebetween, and wherein the interfacial region is spaced apart from the reservoir section.

### CROSS-REFERENCE TO RELATED APPLICATION

This application claims the benefit of priority to PCT/US2023/022150, filed on May 12, 2023, which claims the benefit of priority to U.S. Patent Application No. 63/341,413 filed May 12, 2022, which are both hereby incorporated by reference in their entirety for all purposes.

### TECHNICAL FIELD

This application relates to an atomizer and a method of atomizing a liquid.

### BACKGROUND

Challenges remain for atomizing viscous liquid substances, such as medication, due to inherent viscous properties of the liquid.

### SUMMARY

In one aspect, the present application discloses an atomizer. The atomizer includes a housing. The housing defines a reservoir section. The reservoir section is preferably sized to hold a volume of a liquid in reserve. The atomizer includes at least one piezoelectric element coupled to the housing. The atomizer includes a mesh coupled to the at least one piezoelectric element. The atomizer includes a heater disposed in the housing and outside of the reservoir section, the heater including a heating face oriented substantially opposing to and spaced apart from the mesh, in which the mesh and the heating face define an interfacial region therebetween, and in which the interfacial region is spaced apart from the reservoir section.

In another aspect, the present application discloses a method of atomizing a liquid using the atomizer described above. The method includes: directing a liquid from a reservoir section along a conduit to an interfacial region, the reservoir section being defined by a housing to hold a volume of the liquid in reserve; heating the liquid in an interfacial region with a heater, the interfacial region being defined between a heating face of the heater and a mesh, the mesh being coupled to at least one piezoelectric element of the housing; and exciting at least one piezoelectric element to atomize the heated liquid in the interfacial region spaced apart from the reservoir section.

### BRIEF DESCRIPTION OF DRAWINGS

FIG. 1 is a perspective front view of an atomizer according to an embodiment of the present disclosure;

FIG. 2 is a perspective back view of the atomizer of FIG. 1;

FIG. 3 is a sectional perspective view of the atomizer according to the embodiment of FIG. 1;

FIG. 4 is a sectional perspective view of a piezoelectric device according to an embodiment of the present disclosure;

FIG. 5 is a sectional perspective view of the atomizer according to the embodiment of FIG. 3;

FIG. 6A is a partial sectional view of the atomizer according to the embodiment of FIG. 3;

FIG. 6B is a partial view of a cross-section taken at line A-A.

FIG. 7 is a schematic view of a circuit diagram of an atomizer according to an embodiment of the present disclosure;

FIG. 8 is a plot illustrating the relationship between viscosity and temperature of a liquid mixture of propylene glycol and vegetable glycerin;

FIG. 9 is an exploded view of an atomizer with an interchangeable piezoelectric device according to another embodiment;

FIG. 10 is an exploded view of the piezoelectric device according to one embodiment of the present disclosure;

FIG. 11 is an exploded view of a piezoelectric device according to another embodiment;

FIG. 12 is a sectional exploded view of an atomizer with an interchangeable piezoelectric device according to another embodiment;

FIG. 13 is a partial sectional exploded view of an atomizer with interchangeable piezoelectric device according to another embodiment; and

FIG. 14 is a schematic flow chart of a method of atomizing a liquid.

### DETAILED DESCRIPTION

The terms “atomizer”, “nebulizer”, “vaporizer” and the like may be used interchangeably to refer to a device configured to atomize a liquid, i.e., produce droplets of a liquid such that the droplets are suspended in air or a gaseous medium. Atomizers are useful many applications, including but not limited to the delivery of medicine into the lungs. For the sake of brevity, the term “liquid” is used herein generally, and may refer to a substantially pure substance in a liquid state, a mixture of substances, an aqueous solution, etc. In some non-limiting examples, the liquid to be atomized may be an aqueous liquid such as a mixture of water and medicine. In some examples, the liquid may be a non-aqueous liquid such as propylene glycol, vegetarian glycerin, or a mixture thereof. In the course of an atomizing process, there may be a certain amount of gas present with the liquid. It will be understood that the term “liquid” as used herein may include a substance that is substantially in the liquid state with some gaseous matter mixed therein.

Some substances, including medication, are preferably provided in a glycol solution instead of water. Unfortunately, atomizing a viscous fluid remains a challenge for the conventional atomizers which are typically functional only for atomizing non-viscous liquids with the viscosity of water or close to water. In one aspect, embodiments of the atomizer of the present disclosure addresses the challenges of atomizing viscous liquids. Other advantages will also be apparent from the following disclosure.

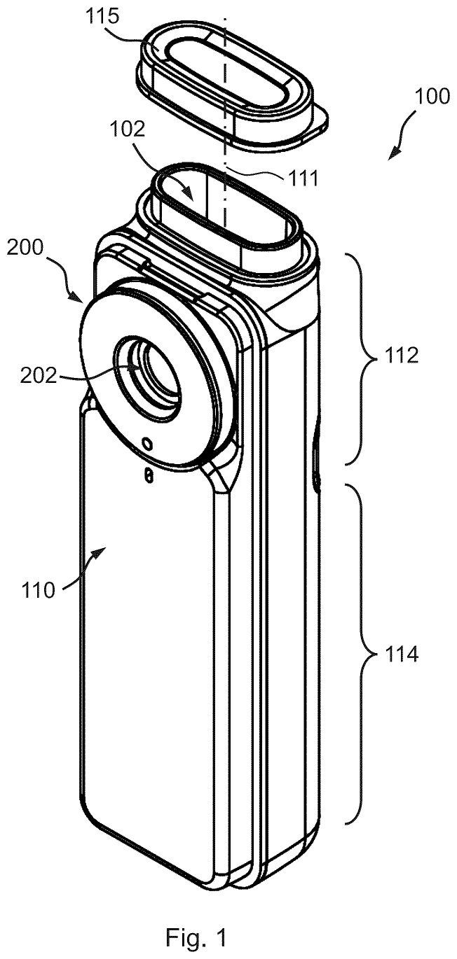

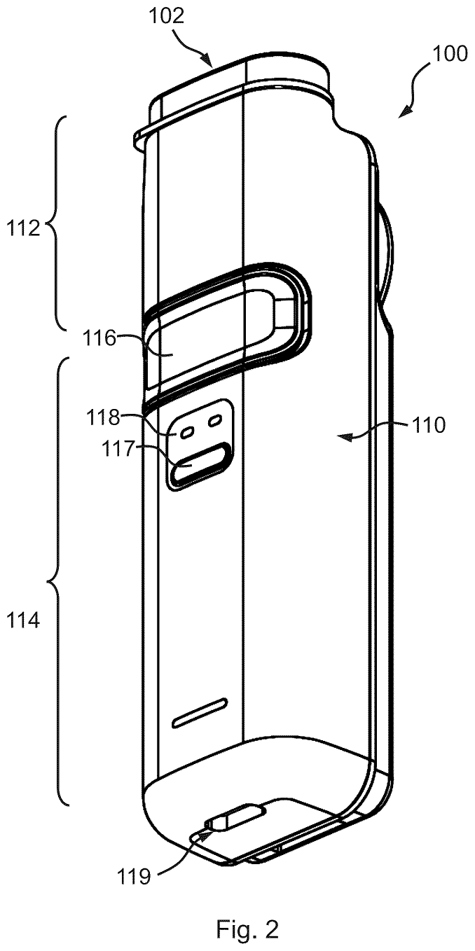

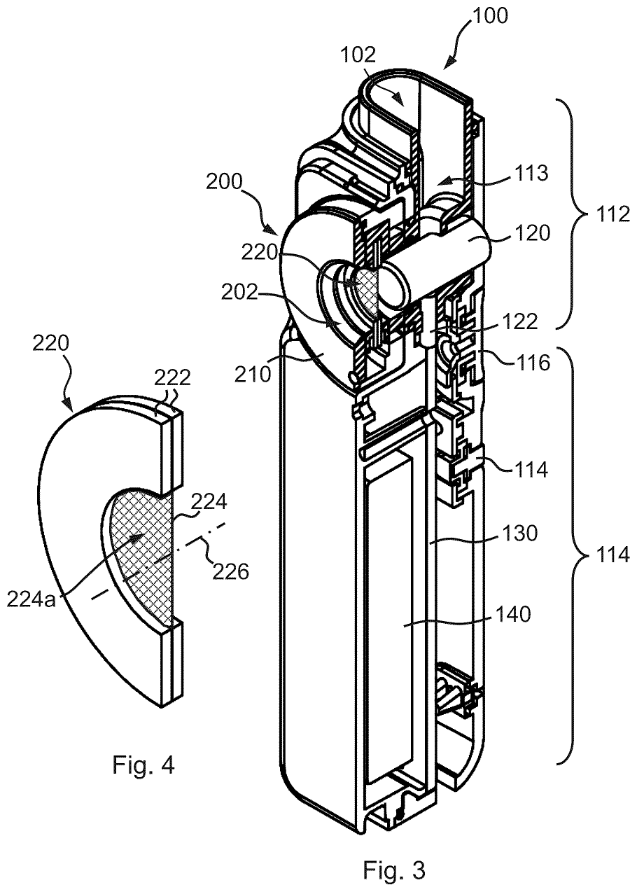

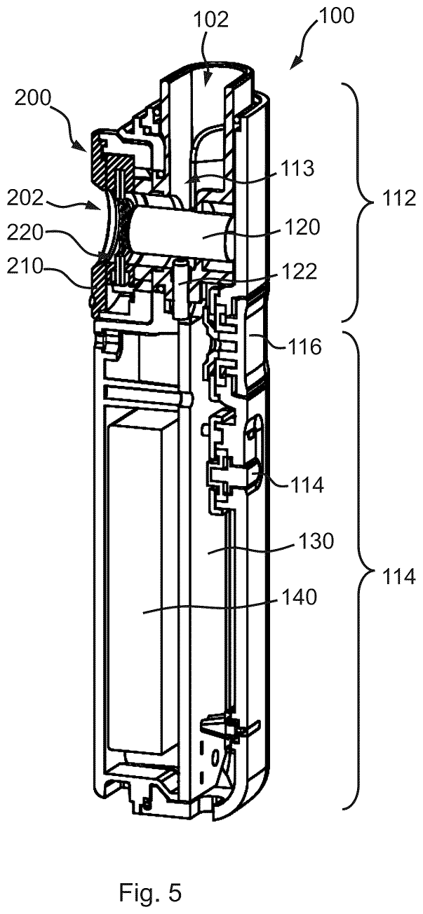

FIGS. 1 to 6 illustrate an embodiment of an atomizer 100 according to the present disclosure. The atomizer 100 is configured such that, in an operating state, the atomizer 100 uses a piezoelectric effect to generate vibrational motion and atomize a liquid. The atomizer 100 includes a housing 110 and a piezoelectric device 200 coupled to the housing 110. The housing 110 may be configured with an elongated form factor defining a longitudinal axis 111. The housing 110 may include an upper portion 112 and a lower portion 114 disposed along the longitudinal axis 111. In some embodiments, the atomizer 100 is a portable device, i.e., configured as a handheld device. In order to facilitate usage and for ergonomic purposes, the lower portion 114 may be configured as a handle suitably sized to be held by a user. The atomizer 100 may include a user interface. The user interface may include at least one of the following: a power button 116, a mode selection switch 117, a display 118, and any combination thereof. A charging port 119 may be provided on the housing 110, for example, near the lower portion 114.

The atomizer 100 includes a chamber 113 configured to receive, and to hold or store a liquid for atomization. The upper portion 112 of the housing may define an inlet 102 leading to the chamber 113, i.e., the inlet 102 is configured to receive a liquid to be held in the chamber 113. A cap 115 may be provided to detachably couple with the inlet 102, such that the inlet 102 may be sealed or at least closed to avoid leakage of liquid from the chamber 113. The chamber 113 may be integrally formed with the housing 110 such that the chamber 113 defines a cavity interior of the housing 110. Alternatively, the chamber 113 may be a separately formed part that is assembled insider the housing 110. The chamber 113 may be fluidly sealed from other parts of the atomizer 100, such as the parts of the atomizer 100 where electronic components are disposed. That is, the chamber 113 may be a cavity or a space disposed in the interior of the housing 110 suitable to receive a liquid.

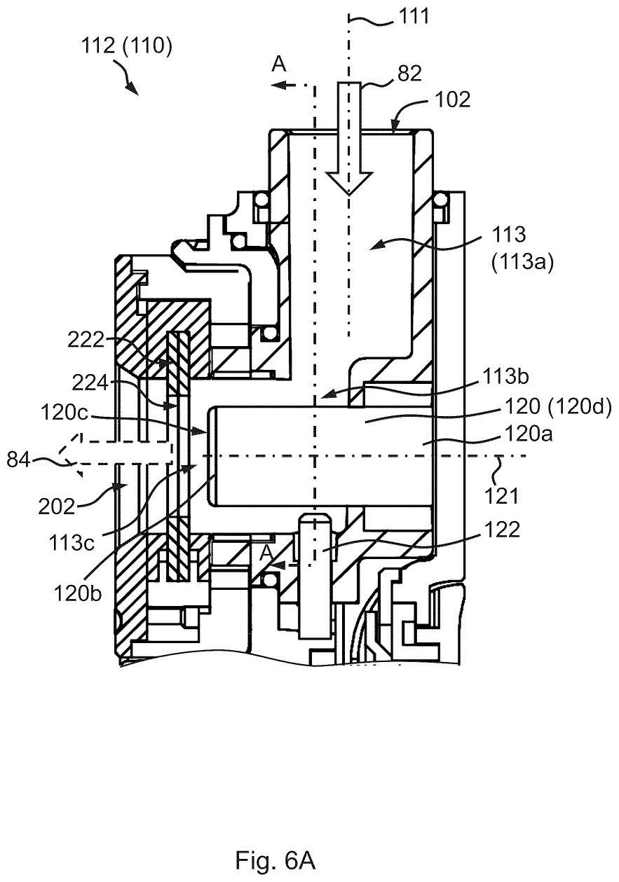

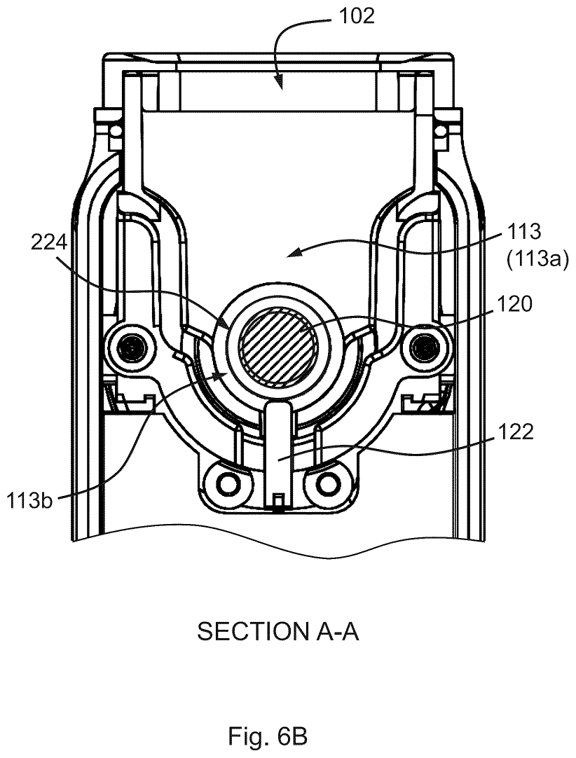

The chamber 113 may include a reservoir section 113a extending from the inlet 102 along the longitudinal axis 111. The chamber 113 may include a conduit 113b in fluid communication with the reservoir section 113a. With reference to the longitudinal axis 111, the reservoir section 113a is preferably proximal to the inlet 102 and the conduit 113b is preferably distal to the inlet 102. The chamber 113 may be configured such that the conduit 113b extends along a transverse axis 121, in which the transverse axis 121 and the longitudinal axis 111 are non-parallel or not parallel to one another. In the non-limiting example illustrated in FIG. 6A, the transverse axis 121 and the longitudinal axis 111 are substantially perpendicular to one another, such that the chamber 113 may be described as an angled chamber.

The atomizer 100 includes a heater 120 disposed in the upper portion 112 of the housing 110. The heater 120 may be disposed in the chamber 113 or at least adjacent to the chamber 113, with the heater 120 configured to heat the contents of the chamber 113. In some examples, the heater 120 is fixed to the housing 110 at a supporting end 120a, with a body of the heater (heating element) extending substantially along the transverse axis 121 to end in a heating end 120b. In the example shown in FIG. 6A, the heater 120 is configured as a substantially straight heating element with a diameter smaller than the diameter or width of the conduit 113b. The heater 120 and the conduit 113b are configured to provide a clearance between the heater 120 and the wall of chamber 113 at the conduit 113b. The clearance may be relatively narrow compared to the dimensions of the reservoir section 113a, such that the reservoir section 113a provides a wide reservoir for liquid flow than the conduit 113b.

In some embodiments, as shown in FIG. 6B, the chamber 113 may include a reservoir section 113a in fluid communication with a conduit 113b, in which the conduit 113 is configured to hold a smaller volume of fluid compared to the reservoir section 113a. The conduit 113b may be defined as a substantially toroidal volume or an annular volume formed around the heater 120. The conduit 113b may be defined between an inner surface and an outer surface, in which the inner surface is formed by the heater shaft 120d of the heater 120, and in which the outer surface is defined by a wall of the chamber 113. The heater shaft 120d may be configured as part of the heating element such that it heats up the liquid flowing along the heater shaft 120d (or flowing along the transverse axis 121) toward the mesh 224 or toward an interfacial region 113c. In operation, only liquid in a part of the chamber 113 is directly heated by the heater 120, and the liquid temperature can be limited. The chamber 113 and the heater 120 are configured to direct the heated liquid toward the piezoelectric-operable mesh 224 (or the interfacial region 113c) to form the atomized product.

The heating end 120b may be configured to define a heating face 120c spaced apart from and substantially opposing a mesh 224. In some examples, the heating face 120c may be a substantially flat surface such that a gap of constant width is defined between the heating end 120b and the mesh 224. The region around the heating end 120b, including the region between the heating face 120c and the mesh 224, is referred to as the interfacial region 113c. The chamber 113 is in fluid communication with the mesh 224 via the interfacial region 113c.

A temperature sensor 122 may be disposed adjacent the heater 120 in the conduit 113b. The temperature sensor 122 may be disposed at a side of the heater 120 distal of the reservoir section 113a. The temperature sensor 122 may be disposed spaced apart from the interfacial region 113c. The temperature sensor 122 is spaced apart from the heater 120 such that the temperature sensed is that of the liquid in the conduit 113b and not that of the heater 120. Preferably, as shown in FIGS. 6A and 6B, the temperature sensor 122 is disposed in the relatively small clearance between a wall of the chamber 113 and the heater 120. In some alternative embodiments, the temperature sensor 122 may be disposed near or in the reservoir section 113a.

The mesh 224 may be part of the piezoelectric device 200. In some embodiments of the present disclosure, the piezoelectric device 200 includes a cap housing 210 defining a central opening corresponding to an outlet 202. The cap housing 210 may be configured to provide a releasable coupling with the housing 110. In some embodiments, the piezoelectric device 200 is detachably or releasably couple-able with the upper portion 112 of the housing 110. The piezoelectric device 200 in operation provides energy to the fluid adjacent the piezoelectric device such that the fluid is atomized. As illustrated, the atomizer 100 is configured to receive liquid from the inlet 102, atomize the liquid via the piezoelectric device 200, and eject or expel the atomized product via the outlet 202.

The piezoelectric device 200 includes a piezoelectric actuator 220 that may be rigidly fixed or supported by the cap housing 210. In some examples, the cap housing 210 may include grooves for rigidly holding the piezoelectric actuator 220. The piezoelectric actuator 220 may include a pair of piezoelectric elements configured to sandwich or clamp a mesh 224 at a perimeter or edge of the mesh 224. In some embodiments, the mesh 224 may be formed of metal. In some embodiments, the pair of piezoelectric elements 222 are disposed at opposing faces of the mesh 224 along an edge of the mesh 224. In one non-limiting example, the pair of piezoelectric elements 222 are configured to rigidly clamp onto a periphery of the mesh 224 such that the periphery of the mesh 224 is substantially stationary relative to the piezoelectric elements 222. The piezoelectric elements 222 may each be configured as a circular disk with a central aperture. The mesh 224 may be described as a piezoelectric-operable mesh in which the piezoelectric elements 222 in operation causes the mesh 224 to vibrate. The vibration may result in agitation of a liquid adjacent or neighboring the mesh 224, i.e., the piezoelectric-operable mesh 224 may transmit vibrational energy to liquid in its vicinity.

In one example, the mesh 224 may be a thin metallic sheet with multiple micro-sized holes or perforations. The perforations form multiple fluid communication paths between the chamber 113 and the outlet 202. For example, each of the perforations may have a diameter in the range of 1 microns (micro-meters) to 10 microns. The perforations may be distributed across the metallic sheet. In another example, the perforations may have a diameter in the range of 2 microns to 5 microns. The diameter or size of the perforations may be determined based on the desired liquid droplet size to be formed. Preferably, the perforations are substantially equally distributed across the metallic sheet, such that an orientation of the metallic sheet relative to the cap housing 210 has no directional effect on the atomization process.

Both the heater 120 and the temperature sensor 122 may be in signal communication with a controller 130, such as a printed circuit board (PCB). The controller 130 may be disposed interior of the housing 110, for example, in the interior of the handle 114. In one example, the controller 130 may be configured to continuously or periodically sense a liquid temperature of the liquid in the chamber 113. For example, the controller may be configured to acquire an analogue signal from the thermistor 122, i.e., sensing a resistance value of the thermistor 122 which would change in response to temperature changes in the liquid temperature. For example, the circuit of the atomizer 100 may be configured with a resistor divider at the controller 130, and instantaneous temperature monitoring may be achieved by detecting the divider voltage. A power source, such as a battery 140, may also be provided interior of the handle 114. In some embodiments, a user may manually define the target temperature using the user interface. The user interface may be configured to permit the user to input the target temperature value, or to select one from multiple predetermined options. The predetermined options may correspond to different liquid compositions, each having a corresponding preferred range of target temperatures.

The piezoelectric elements 222 may be in signal communication with the controller 130, such that the controller 130 may provide a driving voltage/current to induce a vibration of the piezoelectric elements 222 via piezoelectric effect. Vibration of the piezoelectric elements 222 causes a central portion 224a of the mesh 224 to vibrate. That is, the portion not clamped by the piezoelectric elements 222 may be caused to vibrate along a vibration axis 226. The vibration axis 226 may be defined as a normal to a surface of the mesh 224. The vibration 226 may be substantially parallel to the transverse axis 121. The vibration of the mesh 224 causes liquid adjacent to the central portion 224a to atomize and form liquid droplets substantially similar in size to the perforations, e.g., liquid droplets of about 2 microns in diameter. In some examples, the vibration of the piezoelectric elements 222 may be between 80 kHz (kilohertz) to 200 kHz.

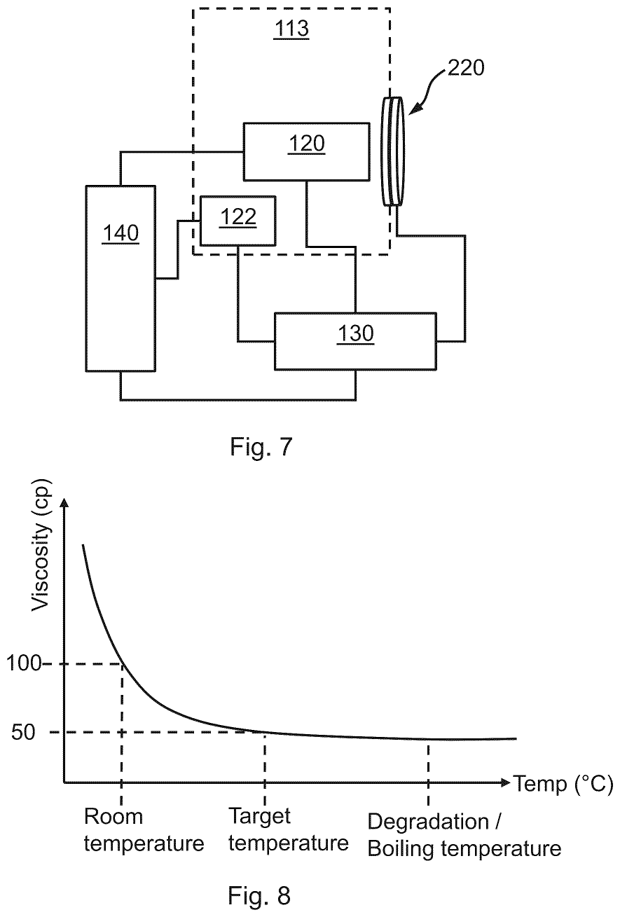

FIG. 7 schematically illustrates one example of a circuit diagram of the atomizer 100. The controller 130 is configured to be in signal communication with each of the heater 120, the temperature sensor 122, and the piezoelectric device 200. The controller 130 may be configured to acquire/receive and/or send signals from/to the respective connected devices. In one example, the heater 120 may be controlled by the controller 130 via a switch. With the input from the temperature sensor 122, the controller 130 controls the switch to toggle between an “ON” state and an “OFF” state. If the switch is “ON”, electrical current flows through the heater 120 to heat up the liquid in the chamber 113. If the switch is “OFF”, no power is delivered to the heater 120 and the liquid in the chamber 113 may cool down. The battery 140 may be configured to provide electrical power to the various parts or components of the atomizer 100, such as the heater 120, the temperature sensor 122, the controller 130, and/or the piezoelectric device 200, etc. In some embodiments, a liquid level sensor is provided to detect a liquid level in the chamber 113. When the liquid level drops below a predetermined threshold, the controller 130 will halt operation for both the heater 120 and the piezoelectric device 200.

One method of atomizing a viscous liquid according to one embodiment of the present disclosure will be described to aid understanding, although it will be understood that present atomizer also works well with non-viscous liquids. A user may choose to introduce a viscous liquid product to be atomized (also referred to as the “liquid”) into the chamber 113 via the inlet 102. The liquid will at least partially fill the chamber 113. Although the user may choose to fill the entire conduit 113b and at least part of the reservoir section 113a with the liquid to be atomized, the atomizer 100 also operates when there is relatively very little liquid in the chamber 113.

In some embodiments of the atomizer 100, the user may choose to switch on the heater 120 without setting a target liquid temperature. In other embodiments, the user may select or input a target liquid temperature. In an operating state, the heater 120 heats up the liquid in the conduit 113b. The temperature sensor 122 is provided adjacent to or neighboring the interfacial region 113c. The chamber 113 is shaped or otherwise configured such that the temperature sensor 122 is disposed to sense the temperature of a relatively small volume of the liquid heated by the heater 120. In other words, the temperature sensor 122 is disposed to measure a temperature of the liquid 82 in the interfacial region 113c or near the interfacial region, without requiring the entire volume of the liquid in the chamber 113 (including the liquid in the reservoir section 113a) to be heated up. By way of the controller 130 acquiring temperature data from the temperature sensor 122 and controlling the heater 120, the atomizer 100 is configured to be responsive to the temperature of the liquid 82 in the interfacial region 113c, and keeps the liquid temperature within a target range of temperature.

In some embodiments, the temperature sensor 122 may be disposed adjacent to the heater 120 and spaced apart from the piezoelectric device 200. In some embodiments, the temperature sensor 122 may be disposed adjacent to the conduit 113b or arranged at a position between the interfacial region 113c and the conduit 113b, such that the liquid 82 in the conduit 113b is heated to a temperature substantially similar to the liquid 82 in the interfacial region 113c. This confers an advantage of having a stable supply of heated liquid 82 in the interfacial region 133c during the process of atomization. The atomizer 100 is configured such that there is no need to heat all the liquid 82 in the reservoir portion 113a or all of the liquid 82 in the chamber 113 to the target temperature. The atomizer 100 is thus configurable with a relatively fast response time.

In some embodiments, the liquid in the conduit 113b is heated by the heater 120 and the conduit 113b serves as a short-term or temporary reservoir for the heated liquid. As the liquid in the interfacial region 113c is atomized and leaves the atomized via the outlet 202, the conduit 113b serves to continuously feed liquid to the interfacial region 113c. The liquid that is fed from the conduit 113b to the interfacial region 113c has been heated in the conduit 113b. This provides a constant or steady supply of heated liquid to the interfacial region 113c during the process of atomization, even as liquid in the interfacial region 113c is constantly being atomized and ejected from the atomizer 100.

In some embodiments, the side(s) of the heater 120 or the heater shaft 120d serves as at least a part of the heating element configured to heat liquid around or next to the heater shaft 120d. In operation, at least some liquid will escape from the interfacial region 113c out of the atomizer 100 via the outlet 202. Liquid in the conduit 113b will tend to flow toward the interfacial region 113c even as the liquid in the conduit 113b is being heated by the heater shaft 120d. The angled configuration in some embodiments of the chamber 113 may facilitate the flow of heated liquid away from the reservoir 113a such that the liquid in the reservoir 113a is less likely to undergo cyclical heating and cooling. This may be preferred in cases where the temperature variations are preferably limited in order to maintain the liquid at a desired quality or condition.

In other embodiments, the side(s) of the heater 120 or the heater shaft 120d may be insulated or otherwise configured such that the heater shaft 120d does not serve as a heating element. That is, the heater 120 may be configured such that only the liquid adjacent the heating end 120c is directly heated by the heater 120, and such that the liquid in the conduit 113b is not directly heated by the heater 120. The conduit 113b serves to at least partially insulate the liquid in the reservoir 113a from the heating effect of the heating end 120b, reducing the likelihood of the liquid in the reservoir 113a and/or the conduit 113b undergoing too many repeated cycles of heating and cooling, reducing or minimizing degradation to the liquid.

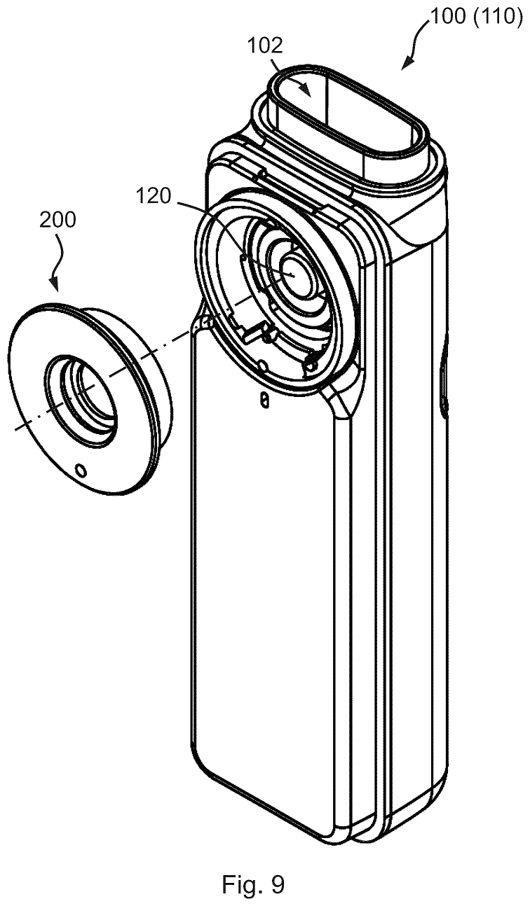

In some embodiments, upon receiving the liquid 82 from the inlet 102, the liquid 82 in the chamber 113 is heated by the heater 120 to a target temperature prior to atomization. In the present disclosure, reference to a “target temperature” may be understood as referring to a temperature within a range of target temperatures, or to a range of target temperatures. The target temperature may be predetermined based on one or multiple parameters of the liquid 82, such as degradation/boiling temperature, fluid viscosity, etc. The target temperature may also be predetermined based on other factors or applications, such as the current room temperature, piezoelectric device output power, intended purpose of atomization, user specific needs, etc. Referring to FIG. 8, without being limited thereto, the target temperature may set higher than room temperature and lower than the degradation/boiling temperature. The controller 130 may monitor the temperature of the liquid 82 in the interfacial region 113c via a closed control loop, including the heater 120, the temperature sensor 122, and the controller 130. The piezoelectric actuator 220 may be concurrently operated, i.e., the mesh 224 may be caused to vibrate. The piezoelectric device 200 causes the liquid in the interfacial region 113c to atomize and form droplets small enough to be ejected through the mesh 225.

In other embodiments of the present disclosure, the user does not need to set a target temperature or target temperature range. After the user switches on the atomizer 100, the piezoelectric device 200 and the heater 120 are concurrently in operation. As the heater 120 raises the liquid temperature (the temperature of the liquid 82) in the conduit 113b, the dynamic viscosity of the liquid in the conduit 113 (including the liquid in the interfacial region 113c) decreases with the increasing temperature until the energy provided by the piezoelectric device 200 atomizes the liquid into droplets small enough to cross the mesh 224 such that an atomized flow 84 of the product is produced at the outlet 202. The user may choose to switch off the atomizer 100 to stop the flow of the atomized product 84. Alternatively, the temperature sensor 122 may be configured to switch off the atomizer upon sensing that the liquid temperature has reached a target temperature.

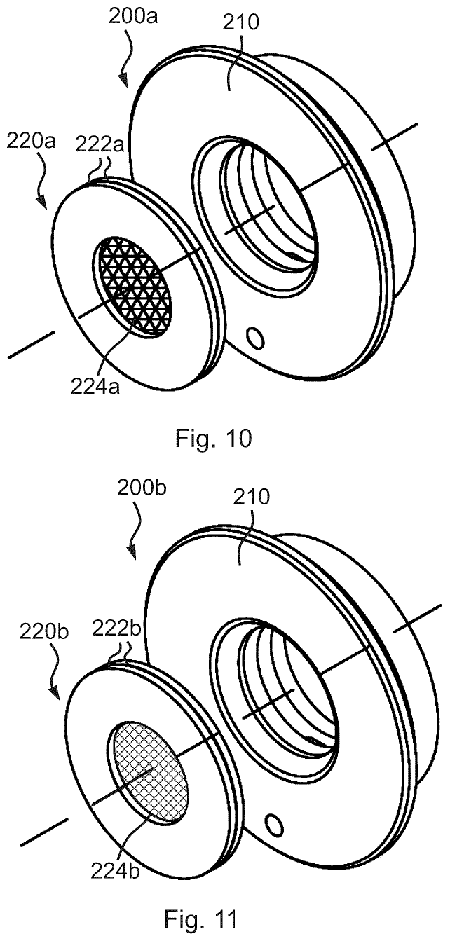

FIGS. 9 to 11 illustrate embodiments of an atomizer 100 with an interchangeable piezoelectric device 200. The piezoelectric device 200 may be configured to be releasably coupled or may be configured to be detachable from the housing 110 for easy replacement, personalization, and/or customization. In some examples, the piezoelectric device 200 may be provided with a bayonet coupling or a magnetic coupling, e.g., to enable a snap-on attachment or detachment. In another example, complementary screw threads may be provided on respective parts of the piezoelectric device 200 and the housing 110. As illustrated in FIGS. 10 and 11, the same atomizer 100 may receive different piezoelectric devices 200a/200b, where each of the piezoelectric devices 200a/200b may include a similar cap housing 210, but has different piezoelectric actuators 220a/220b. Each of the piezoelectric actuator 220a/220b may be configured with a different mesh 224a/224b. The meshes 224a/224b are configured with different perforation sizes. In some embodiments, the piezoelectric device 200 may be in signal communication with the controller 130 such that a predetermined target temperature suitable for the perforation size is communicated to the controller 130. In some applications, the user may select a mesh 224 according to the type of product to be atomized. In some other applications, the user may select a mesh 224 according to whether the atomized product is intended to be exhaled after inhalation. In yet other applications, the user may select a mesh 224 that produces an atomized product characterized by a mean droplet size that facilitates retention of the atomized product in the respiratory system after inhalation.

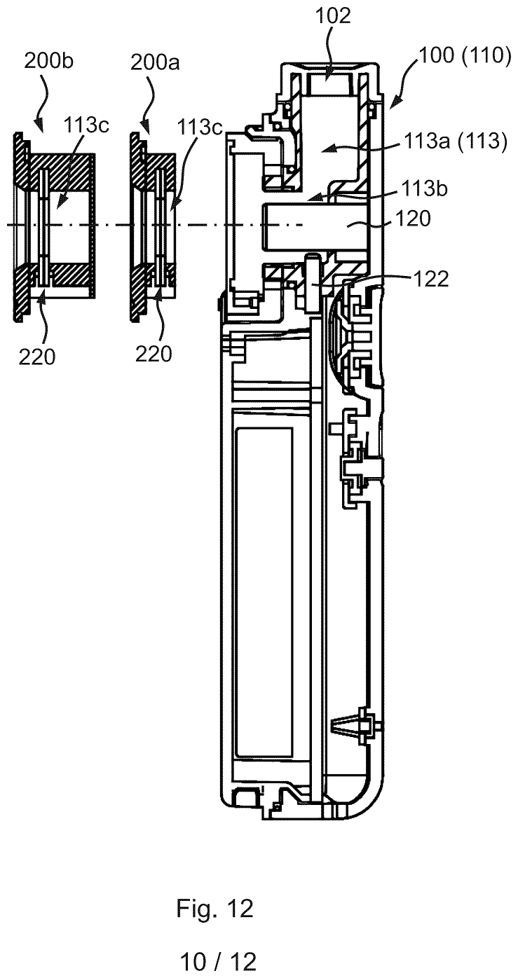

Referring to FIG. 12, in another embodiment of an atomizer 100 with an interchangeable piezoelectric device 200, the interfacial region 113c of the chamber 113 may be fully or at least partial defined by the piezoelectric device 200. In other words, the size of the interfacial region 113c and hence chamber 113 is variable or selectable according to different piezoelectric devices 200. By selecting a piezoelectric device 200 with a larger pre-atomizing chamber 113c enables a larger volume of liquid to be held in the fluid chamber 113. This provides the benefit for long duration or high atomization rate applications. It may be appreciated that a chamber 113 with variable/selectable volume may also facilitate dosage prescription, such that the user may simply fill up the chamber 113 and seal it with a cap, without the need for measuring the liquid volume for use.

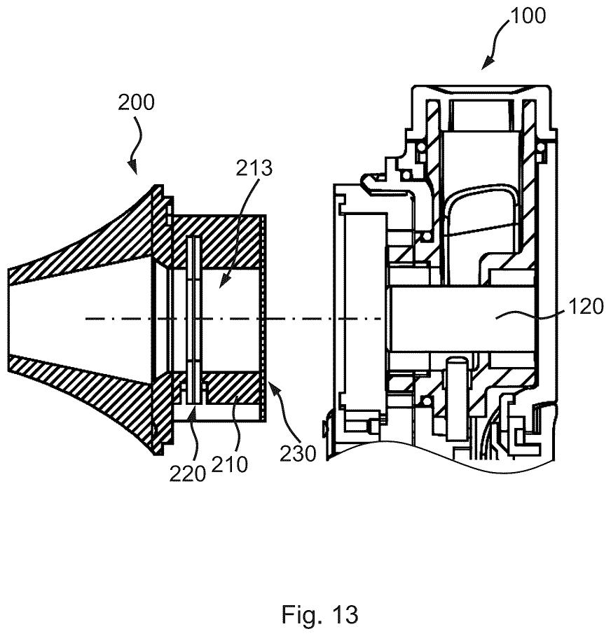

In another embodiment of an atomizer 100 with an interchangeable piezoelectric device 200, referring to FIG. 13, the piezoelectric device 200 may be configured as a cartridge provided with a sealed chamber 213 filled with liquid for atomization. The chamber 213 may be enveloped by a cap housing 210 defining a through aperture, with a thermal conductive membrane 230 on one end of the aperture and the piezoelectric actuator 220 on the other end of the aperture. Due to the small size of the perforations, in which a pressure gradient is required for liquid to pass through, the liquid in the sealed chamber 213 are substantially limited from escaping from the sealed chamber 213. The piezoelectric device 200 may be in signal communication with the controller 130 such that a predetermined target temperature suitable for the liquid in the sealed chamber 213 and the perforation size is communicated to the controller 130. In this embodiment, the sealed chamber 213 will be brought into contact with the heater 120 and the temperature sensor 122 upon attachment of the piezoelectric device 200 with the housing 110. The sealed chamber 213 may be configured to prohibit fluid communication with the housing 110, i.e., due to the membrane 230. However, the membrane 230 permits thermal contact of the fluid in the sealed chamber 213 with the heater 120 for heating. A temperature sensor may also be provided in thermal contact with the membrane 230 such that the temperature of the fluid in the sealed chamber 213 may be sensed. This advantageously provides a contamination free use, whereby the same atomizer 100 may be used with different piezoelectric devices 200 without the need for through cleaning/sterilizing.

In another aspect of the disclosure, a method for atomizing a liquid is provided. The method includes heating a liquid in a chamber until an atomized form of the liquid is produced. The method includes controlling the heating of the fluid such that the liquid temperature is kept lower than a degradation temperature of the liquid.

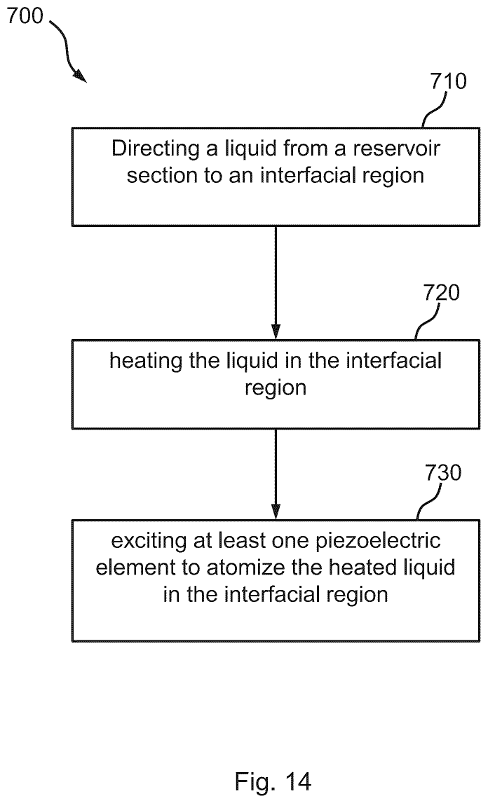

According to an embodiment as illustrated in FIG. 14, a method 700 of atomizing a liquid includes: in step 710, directing a liquid from a reservoir section to an interfacial region, the interfacial region being spaced apart from the reservoir section; in step 720, heating the liquid in the interfacial region, the interfacial region disposed between a mesh of a piezoelectric device and a heating face of the heater; and in step 730, exciting at least one piezoelectric element of the piezoelectric device to atomize the heated liquid in the interfacial region.

In some embodiments, the method 700 may further include sensing a temperature of the liquid in the interfacial region. In some embodiments, the method 700 may further include controlling the heater based on the temperature of the liquid in the interfacial region and a target temperature. In some embodiments, the method 700 may further include exciting the at least one piezoelectric element response to the temperature of the liquid in the interfacial region reaches the target temperature.

In some embodiments, the method 700 may further include heating up a liquid in a conduit of a chamber with a heater; and providing heated liquid from the conduit to the interfacial region. In some embodiments, the method 700 may further include sensing a temperature of the liquid in the conduit. In some embodiments, the method 700 may further include replacing the piezoelectric device, wherein the piezoelectric device is releasably coupled to the housing.

Alternatively described, the present atomizer 100 includes a housing. The housing defines a reservoir section 113a. The reservoir section 113a is preferably sized to hold a volume of a liquid in reserve. The atomizer 100 includes at least one piezoelectric element 222 coupled to the housing. The atomizer 100 includes a mesh 224 coupled to the at least one piezoelectric element 222. The atomizer 100 includes a heater 120 disposed in the housing and outside of the reservoir section 113a, the heater 120 including a heating face 120c oriented substantially opposing to and spaced apart from the mesh 224, in which the mesh 224 and the heating face 120c define an interfacial region 113c therebetween, and in which the interfacial region 113c is spaced apart from the reservoir section 113a.

Preferably, the at least one piezoelectric element 222 when excited is configured to vibrate the mesh 224 along a vibration axis, in which the heating face 120c is spaced apart from the mesh 224 along the vibration axis.

The atomizer 100 may further include a conduit 113b in fluid communication with the reservoir section 113a and the interfacial region 113c. The reservoir section 113a preferably extends from an inlet 102 along a longitudinal axis, with the conduit 113b extending along a transverse axis, and with the transverse axis and the longitudinal axis being non-parallel to one another. The transverse axis is preferably substantially parallel to the vibration axis. The heater 120 is preferably disposed in the conduit 113b such that the heater 120 and the conduit 113b are substantially coaxial to define an annular fluid communication path leading to the interfacial region 113c.

The conduit 113b is configured to hold a smaller volume of the liquid compared to the reservoir section 113a. In operation, the liquid in the conduit 113b is heated by the heater 120, and the conduit 113b is configured to continuously feed heated liquid to the interfacial region 113c.

The atomizer 100 may further include a temperature sensor disposed spaced apart from the heater 120 and adjacent to the conduit 113b to sense a temperature of the liquid in the conduit 113b. The conduit 113b at least partially insulates the liquid in the reservoir section 113a from a heating effect of the heater 120. The atomizer 100 may further include a temperature sensor disposed adjacent to the interfacial region 113c to sense a temperature of the liquid in the interfacial region 113c. The heating face 120c is preferably a substantially flat surface, such that a gap of substantially constant width is defined between the heating face 120c and the mesh 224.

The at least one piezoelectric element 222 may include a pair of piezoelectric elements, in which a periphery of the mesh 224 is clamped between the pair of piezoelectric elements 222. Optionally, the atomizer 100 includes a cap housing 210 that is releasably coupled to the housing. The atomizer 100 may further include a controller 130 disposed interior of the housing, in which the controller 130 is configured to sense a liquid temperature of the liquid in the reservoir section 113a and to controllably drive (excite) the at least one piezoelectric element responsive to the liquid temperature.

The present disclosure describes a method of atomizing a liquid using the atomizer 100 described above. The method includes: directing a liquid from a reservoir section 113a along a conduit 113b to an interfacial region 113c, the reservoir section 113a being defined by a housing to hold a volume of the liquid in reserve; heating the liquid in an interfacial region 113c with a heater, the interfacial region 113c being defined between a heating face of the heater 120 and a mesh 224, the mesh 224 being coupled to at least one piezoelectric element 222 of the housing; and exciting at least one piezoelectric element 222 to atomize the heated liquid in the interfacial region 113c spaced apart from the reservoir section 113a.

The method may further include sensing a temperature of the liquid in the interfacial region 113c. The method may further include controlling the heater 120 based on the temperature of the liquid in the interfacial region 113c and a target temperature. The method may further include exciting the at least one piezoelectric element 222 in response to the temperature of the liquid in the interfacial region 113c reaches the target temperature. The method may further include heating a liquid in the conduit 113b with the heater 120 such that the liquid arrives at the interfacial region 113c at least partially heated to the target temperature. The method may further include sensing a temperature of the liquid in the conduit 113b.

Prototypes of the present atomizer 100 have successfully demonstrated the ability to produce an atomized form of a viscous liquids. By interchanging the mesh 224/piezoelectric device 200, different droplet sizes could be produced. In some experiments, a liquid mixture of propylene glycol and vegetable glycerin was used. Such a mixture has a viscosity of 100 cp (centipoise) at room temperature (water as a viscosity of 0.0091 cp at room temperature). Previously, using conventional atomizers, it had not been possible to produce an atomized form of such a viscous liquid mixture having a viscosity of several orders larger than that of water. While there may have been earlier attempts to heat the product to be atomized, the conventional devices had to deal with degradation of the product as it is overheated or after repeated heating cycles. In contrast, the present atomizer could produce a desired flow of the atomized liquid mixture within a safe temperature, with the atomized product being characterized by a desired droplet size. For example, in the case of a product in a medium of propylene glycol and/or vegetable glycerin, a desired atomized product could be produced at a liquid temperature of about 80 degrees Celsius, whereupon the atomizer is configured to prevent further heating of the liquid. The present atomizer opens up new ways for drug delivery, especially for medicines which need to be kept in a non-aqueous mixture/solution. Even medicinal products with bacteria or cell cultures which may denature or degrade at high temperatures can be safely delivered using the present atomizer.

It will be understood that the described features, structures, or characteristics may be combined in any suitable manner in one or more embodiments. In the foregoing description, numerous specific details are provided to give a thorough understanding of embodiments. One skilled in the relevant art will recognize, however, that the various embodiments can be practiced without one or more of the specific details, or with other methods, components, materials, etc. In other instances, some or all known structures, materials, or operations may not be shown or described in detail to avoid obfuscation.

As used herein, the singular “a” and “an” may be construed as including the plural “one or more” unless clearly indicated otherwise. Reference throughout this specification to “one embodiment”, “another embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

1. An atomizer comprising:
a housing defining a reservoir section, the reservoir section being sized to hold a volume of a liquid in reserve;
at least one piezoelectric element coupled to the housing;
a mesh coupled to the at least one piezoelectric element; and
a heater disposed in the housing and outside of the reservoir section, the heater including a heating face oriented substantially opposing to and spaced apart from the mesh,
wherein the mesh and the heating face define an interfacial region therebetween, and wherein the interfacial region is spaced apart from the reservoir section.

2. The atomizer as recited in claim 1, wherein the at least one piezoelectric element is configured to vibrate the mesh along a vibration axis, wherein the heating face is spaced apart from the mesh along the vibration axis.

3. The atomizer as recited in claim 1, further comprising a conduit in fluid communication with the reservoir section and the interfacial region.

4. The atomizer as recited in claim 3, wherein the reservoir section extends from an inlet along a longitudinal axis, and wherein the conduit extends along a transverse axis, the transverse axis and the longitudinal axis being non-parallel to one another.

5. The atomizer as recited in claim 4, wherein the transverse axis is substantially parallel to the vibration axis, and wherein heater is disposed in the conduit such that the heater and the conduit are substantially coaxial to define an annular fluid communication path leading to the interfacial region.

6. The atomizer as recited in claim 3, wherein the conduit is configured to hold a smaller volume of the liquid compared to the reservoir section.

7. The atomizer as recited in claim 3, wherein the liquid in the conduit is heated by the heater, and the conduit is configured to continuously feed heated liquid to the interfacial region.

8. The atomizer as recited in claim 7, further comprising a temperature sensor disposed spaced apart from the heater and adjacent to the conduit to sense a temperature of the liquid in the conduit.

9. The atomizer as recited in claim 3, wherein the conduit at least partially insulates the liquid in the reservoir section from a heating effect of the heater.

10. The atomizer as recited in claim 1, further comprising a temperature sensor disposed adjacent to the interfacial region to sense a temperature of the liquid in the interfacial region.

11. The atomizer as recited in claim 1, wherein the heating face is a substantially flat surface, such that a gap of substantially constant width is defined between the heating face and the mesh.

12. The atomizer as recited in claim 1, wherein the at least one piezoelectric element comprises a pair of piezoelectric elements, and wherein a periphery of the mesh is clamped between the pair of piezoelectric elements.

13. The atomizer as recited in claim 1, further comprising a cap housing, wherein the cap housing is releasably coupled to the housing.

14. The atomizer as recited in claim 1, further comprising a controller disposed interior of the housing, wherein the controller is configured to sense a liquid temperature of the liquid in the reservoir section and to controllably drive the at least one piezoelectric element responsive to the liquid temperature.

15. A method of atomizing a liquid using the atomizer of claim 1, the method comprising:
directing a liquid from a reservoir section along a conduit to an interfacial region, the reservoir section being defined by a housing to hold a volume of the liquid in reserve;
heating the liquid in an interfacial region with a heater, the interfacial region being defined between a heating face of the heater and a mesh, the mesh being coupled to at least one piezoelectric element of the housing; and
exciting at least one piezoelectric element
to atomize the heated liquid in the interfacial region spaced apart from the reservoir section.

16. The method as recited in claim 15, further comprising sensing a temperature of the liquid in the interfacial region.

17. The method as recited in claim 16, further comprising controlling the heater based on the temperature of the liquid in the interfacial region and a target temperature.

18. The method as recited in claim 17, further comprising exciting the at least one piezoelectric element in response to the temperature of the liquid in the interfacial region reaches the target temperature.

19. The method as recited in claim 18, further comprising heating a liquid in the conduit with the heater such that the liquid arrives at the interfacial region at least partially heated to the target temperature.

20. The method as recited in claim 19, further comprising sensing a temperature of the liquid in the conduit.
